(19) United States
(12) Patent Application Publication
(10) Pub. No.: US 2019/0046204 A1
(43) Pub. Date: Feb. 14, 2019

(54) PROSTHESIS FOR INGUINAL HERNIAS

(71) Applicant: Felix CHECA AYET, Valencia (ES)

(72) Inventor: Felix CHECA AYET, Valencia (ES)

(21) Appl. No.: 16/046,404

(22) Filed: Jul. 26, 2018

(30) Foreign Application Priority Data
Aug. 13, 2017 (ES) 20170030968U

(51) Int. Cl. A61B 17/12

(52) U.S. Cl. CPC A61B 17/12013; A61B 2017/00477

(57) ABSTRACT

A prosthesis for inguinal hernias which is surgically inserted in a patient, made of a sole flat piece with a central cutting line which divides the flat piece into two asymmetric sides helically foldable to each other. Each asymmetric side has an oval shape with a narrow separation at middle point on its external edge due to an external cleft to provide an upper oval area and a lower oval area. After folding, the two sides form a reinforced central hole to allow the spermatic cord to fit therethrough. A preferably conic-shaped cap is provided in one of the lower oval areas, whose larger diameter base is fixed to the flat surface of one of the asymmetric sides and its lateral walls are free and adhered to the internal tissues of the patient.

### CROSS-REFERENCE TO RELATED APPLICATIONS AND CLAIM TO PRIORITY

This application is related to Patent Application No. 20170030968U filed Aug. 13, 2017, in Spain, the disclosure of which is incorporated herein by reference and to which priority is claimed.

### FIELD OF THE INVENTION

This invention relates to a prosthesis for the treatment or surgical repair of inguinal hernias. The prosthesis contains a sole flat piece divided into two connected asymmetrical halves which are foldable into a helically twisted configuration. A cap is also provided as an integral part of the prosthesis.

### BACKGROUND OF THE INVENTION

Inguinal hernia surgery is a surgical operation which has been considerably developed over the years, both regarding surgical techniques by using meshes to repair inguinal hernias and regarding the development of biomaterials for its use, always looking for an abdominal-pressure resistant solution.

In this sense, the use of a prosthesis is well-known in this sector of medicine. Generally, these prostheses are made of materials such as polypropylene. Even though they are made in different shapes, they do not reach the effectiveness level of this invention. Nowadays, it is common to use the Rutkow technique in which a flat mesh is placed in the upper part of the defect. This technique is based on a stress-free repair which consists of placing a reinforcement mesh in that area. Taking into account this aspect, EP01931800.5, which develops a prosthesis with a hollow tapered shape, and EP00901329, which develops a multi-layer prosthesis made of a multiplicity of layers which are connected to each other, stand out as background within the technique status. The present invention differs from this background as it is made of a sole flat piece, which improves and simplifies these known types of prostheses.

At this point, EP1852086 of the same applicant of this invention stands out. It could be considered as the closest registration in the technique status which, even though it presents a sole and flat prosthesis, the new invention defines a central hole or orifice like the prior one and also adds a cap which is part of the prosthesis. In addition, in the Rutkow technique some cone-shaped or umbrella-shaped caps are used in such a way that these prostheses are made of an external folded layer combined with an internal eight-petal mesh ring whose unfolded configuration adheres by itself to the deepest side of the abdominal wall like a minor circumference. This technique and type of prosthesis differs from this invention because the Rutkow technique uses a flat prosthesis which does not allow a double reinforcement, it is a methodology that uses at least two different units and which does not ensure an accurate fastening of the cap, which may scroll around the body.

Then, with the prosthesis for inguinal hernias of this invention we solve in a single piece the problems of known prostheses, as this single piece provides reinforcement and resistance areas in the affected area, while and the cap is prevented from moving once it is in place.

Taking into account the solutions before defined, no prior prosthesis included a cap as part of its own structure. Thus, with the prosthesis hereinafter detailed, we go a step forward in the inguinal surgery field as it presents a structure which is able to protect the sensitive and weak areas of the abdominal walls, increasing safety due to its immobility and position once it is surgically placed.

Subsequently, a detailed description of the invention is provided which completes the general ideas included in this section.

### SUMMARY OF THE INVENTION

The prosthesis which is the subject matter of this invention contains a flat piece of material having a middle dividing line which divides the flat piece into two asymmetrical halves or sides. When the halves are twisted with a helical movement or when the halves are folded one over the other, they form a central hole which provides two areas with double resistance and reinforcement. The prosthesis further has a cap fixed to one of the two asymmetrical halves, which ensures fixation of the prosthesis when it is placed in the area of action.

In particular, and as an example of the creation of such prosthesis, the flat piece contains two halves divided by a middle dividing line. The two halves are then overlapped, for example, the right half over the left half, by lacing and overlapping a right lower tab over the left lower tab. A cap is fixed on the upper side of one of the asymmetrical halves. Thus, the spermatic cord, whose back wall is to be reinforced, as well as the anatomic ring through which such cord rises, are wrapped with the first half of the prosthesis, resulting in a reinforcement area which corresponds to the anatomic areas whose alteration provokes the appearance of the most common and frequent shapes of hernias is increased. Therefore, the handling of such prosthesis consists of folding one asymmetric half over the other, in such a way that a central hole is generated. The overlap of the asymmetric halves provides a doubly protected and reinforced area.

At this point, and in order to improve that central open area, the middle dividing line does not completely cut the flat piece, but leaves an upper area of continuity between the two halves, with an open groove having sufficient flexibility to allow the folding movement of the two asymmetric sides. Each of the asymmetric sides has preferably a double oval shape (an upper oval area and a lower oval area), and the oval areas are separated by a narrow area having an external edge where a cleft is made. Thus, the upper oval area allows turnings or helical movements, whereas the lower oval area, as a lower fin, is an elongated body which allows closing the set at the turning. The cleft allows generating and reinforcing the central open area once the set is folded.

A cap is fixed to one of the edges of the lower oval area or lower fin. The cap may be made of the same material as the prosthesis and which provides a protrusion from the flat piece. Preferably, the cap is conically or convexly shaped, although it may also have a tapered shape or another similar shapes, such that the shape contains a larger base or diameter linked or coupled to the flat surface of the lower oval area and a smaller peak or diameter is freely protruding away from the flat surface.

The present invention provides the cap as a single unitary piece with the rest of the prosthesis, which prevents the cap from scrolling and moving around in the patient's body when in use. Whether the cap scrolls around the body, the reinforcement of the abdominal wall is not properly fulfilled, resulting in risks for the patient's health of having a foreign body scrolling and moving around in his/her body.

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain the principles of the invention. In such drawings:

FIG. 1 is a depiction of an elevational view of the prosthesis set surrounding the spermatic cord;

FIG. 2 is a perspective depiction of the prosthesis surrounding the spermatic cord, seen from a lower point and according to the first figure;

FIG. 3 is a depiction of the prosthesis which is the subject matter of this invention in a horizontal plane seen from a lower or posterior point;

FIG. 4 is a depiction of a posterior horizontal view of the prosthesis in which we can see the first overlapping phase of the two asymmetric sections;

FIG. 5 is a depiction, continuing with the prior figure, in which we can see the completion of the overlapping of the two asymmetric sections; and

FIG. 6 is the depiction of a posterior horizontal view of the completely folded prosthesis with its reinforcement areas in which we can see the cap placement.

### DETAILED DESCRIPTION OF THE PREFERRED EMBODIMENT(S)

We must say that the following is the explanation of the drawings and at the same time it can be used as an explanation of a preferential way to put the invention into practice.

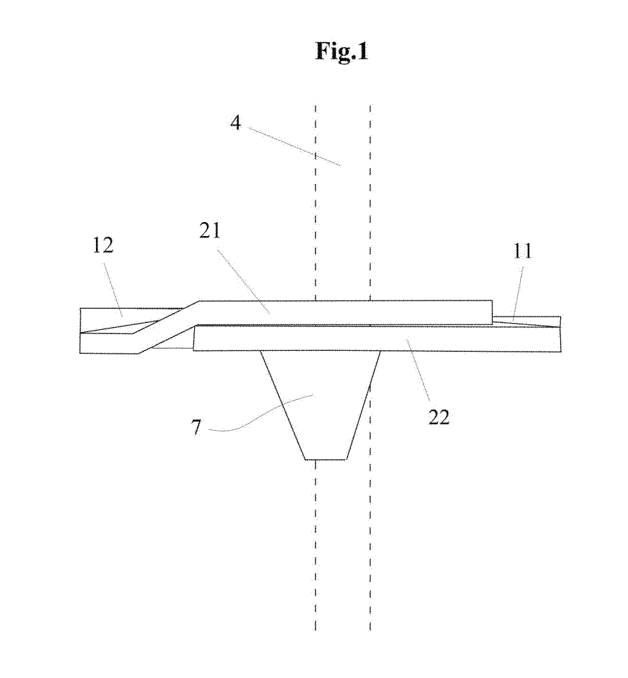

As best shown in the FIGS. 1 and 2, the inguinal prosthesis is a flat piece of material which contains two asymmetrical sides or halves (1, 2) with rounded edges that are helically intertwined into a twisted configuration to generates a central, preferably circular- or oval-shaped, opening (3) through which a spermatic cord (4) is inserted. The prosthesis provides two areas of double resistance around the central opening (3) and a cap (7) fixed on a lower tab of one of the two halves (1, 2).

In particular, FIG. 1 shows an elevation view of the inguinal prosthesis wrapping the spermatic cord (4) which goes through the central cleft (3) generated by the helical twisted folding of the two asymmetric sides (1, 2) of the prosthesis. The cap (7) is fixed in one of the lower oval areas or lower fin (22), which is described below.

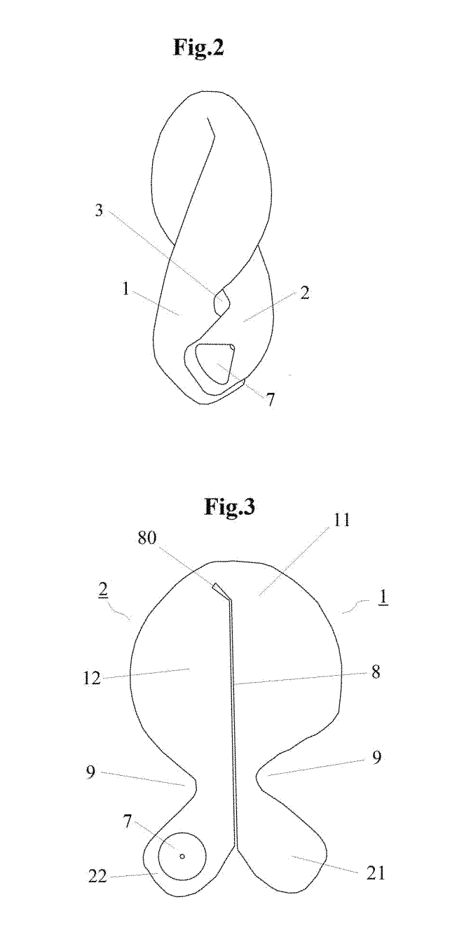

FIG. 3 shows the unfolded prosthesis in a posterior horizontal plane or seen from a lower position, in which you can see the two asymmetric sides (1, 2) and the central dividing line (8), where the dividing line (8) does not completely divide the two haves (1, 2) but provide a linking area connecting the two asymmetric halves (1, 2). The dividing line (8) has a small groove (80) being opened and bent at an angle to a straight part of the cutting line (8) to allow the two asymmetric sides (1, 2) to be folded to each other. The two asymmetric sides (1, 2) have rounded peripheral edge lines, in such a way that do not generate corners or areas which may cause pain to the patient when in use. As shown in the figures, it is preferable that each asymmetric side has a generally ovalular shape, with a narrow separation at middle portion thereof to divide the asymmetric side into an upper oval area (11, 12) and a lower oval area (21, 22). The middle portion contains an external edge forming a cleft (9) in such a way that provides the upper oval area (11, 12) to allow turnings or helical twisting of the prosthesis. The lower oval area (21, 22) provides an elongated, oblong fin or a lower tab to close and lock the helical twisting or folding of the two asymmetric sides. The exterior cleft (9) provides a reinforced the central opening (3) when the prosthesis is properly folded. In a portion of the lower oval area (21), the cap (7) is placed and fixed thereto. The cap (7) is a cone-shaped element, whose larger base or diameter is fixed to the lower oval area (21) and tip or smaller diameter extends away from the lower oval area (21). Thus, with a single prosthesis it is possible to provide double reinforcement and movement resistance. By including the cap (7) as part of the prosthesis, it is possible to avoid the prosthesis from becoming detached or scrolling around inside the body of the patient when in use.

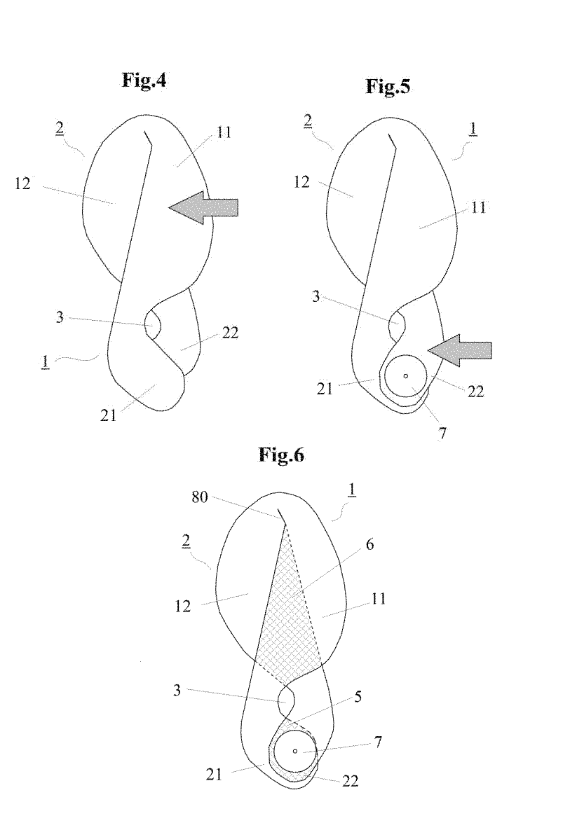

FIGS. 4 to 6 disclose the proper folding method of the asymmetric sides (1, 2) of the prosthesis. First, the asymmetric side (1) without the cap (7) is placed overlapping the asymmetric side (2) with the cap (7) on its lower oval area (22). To finish the folding of the prosthesis, the lower oval area (22) with the cap (7) (of asymmetric side (2)) is pulled over the lower oval area (21) without the cap (7) (of asymmetric side (1)) to lock the prosthesis in a helically twisted position. When properly folded, the upper oval area 12 lays below the upper oval area 11, while the lower oval area 22 (with the cap (7)) lays over the lower oval area 21. The lower oval areas (21, 22) overlapped to provide a double reinforcement area (5). The central opening (3) is generated with a circular or oval shape. A double reinforcement area (6) (see FIG. 6) is provided by overlapping upper oval areas (11, 12). The cap (7) is provided integral to the prosthesis. The arrows drawn in FIGS. 4 and 5 show the folding movement of the two prior overlapping stages.

The prosthesis design is based on the normal anatomical structure of the human body and is aimed to generate a helically laced structure with a protuberance as a variable-shaped cap which aligns with and solves the two usually weak anatomical areas in inguinal hernias.

The inguinal prosthesis is preferably made of a polymer, more preferably polypropylene. The cap (7) is preferably made of the same material as the prosthesis and is integral with the prosthesis. For example, the cap (7) may be molded as a feature during the formation of prosthesis.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A prosthesis for treatment of the surgical treatment of inguinal hernia, comprising:
a. a flat piece of material containing two connecting asymmetric halves configured to be foldable into a helical twisted configuration to provide a central opening; and
b. a cap integrally fixed to a bottom of one of the two connecting asymmetric halves.

2. The prosthesis of claim 1, wherein the halves are divided by a central dividing line.

3. The prosthesis of claim 2, wherein the central dividing line contains an opened groove at one end, the groove being bent at an angle to a straight part of the dividing line.

4. The prosthesis of claim 1, wherein the asymmetric halves contain rounded peripheral edges.

5. The prosthesis of claim 1, wherein each of the asymmetric halves contains an upper oval area and a lower oval area divided from each other by a cleft.

6. The prosthesis of claim 5, wherein the cap is fixed to the lower oval area of one of the asymmetric halves.

7. The prosthesis of claim 5, where in the cap is conical in shape.

8. The prosthesis of claim 7, wherein the cap contains a base fixed to the lower oval area and a tip protruding away from the lower oval area.

9. The prosthesis of claim 7, wherein the lower oval areas of the halves provide lower tabs to lock the helical twist configuration in place when the asymmetric halves are folded.

10. The prosthesis of claim 5, wherein when folded, the upper oval areas provide a first overlap and the lower oval area provide a second overlap.

11. A prosthesis for treatment of the surgical treatment of inguinal hernia, comprising:
a. a piece of material containing two connecting asymmetric halves folded into a helical twisted configuration to provide a central opening; and
b. a cap integrally fixed to a bottom of one of the two connecting asymmetric halves.

12. The prosthesis of claim 10, wherein each of the asymmetric halves contains an upper oval area and a lower oval area divided from each other by a cleft.

13. The prosthesis of claim 12, wherein the clefts are joined form the central opening.

14. The prosthesis of claim 12, wherein the lower oval areas provide overlapping lower tabs to lock the helical twisted configuration in place and to provide a first reinforced area bordering the central opening.

15. The prosthesis of claim 14, wherein the upper oval areas overlap each other to provide a second reinforced area bordering the central opening.

16. The prosthesis of claim 15, wherein the cap is conical in shape.

17. The prosthesis of claim 12, wherein the cap contains a base fixed to the lower oval area of one of the asymmetric halves and a tip protruding away from the lower oval area.

18. The prosthesis of claim 11, wherein the asymmetric halves contain rounded peripheral edges.

19. A method for the surgical treatment of inguinal hernia, comprising the steps of
a. providing the prosthesis of claim 1;
b. folding the prosthesis into a helical twisted configuration to provide a central opening; and
c. fitting a spermatic cord through the central opening.

20. The method of claim 17, further comprising the step of aligning the cap with the weak anatomical areas of inguinal hernias.
